(19) United States
(12) Patent Application Publication
(10) Pub. No.: US 2017/0166869 A1
(43) Pub. Date: Jun. 15, 2017

(54) BIOLOGICALLY OPTIMIZED ADULT MESENCHYMAL STEM CELLS

(71) Applicant: Jose Javier Lopez GONZALEZ, Chula Vista, CA (US)

(72) Inventor: Jose Javier Lopez GONZALEZ, Chula Vista, CA (US)

(21) Appl. No.: 15/116,805

(22) PCT Filed: Feb. 4, 2015
(86) PCT No.: PCT/US15/14498

§ 371 Date: Aug. 4, 2016

Related U.S. Application Data
(60) Provisional application No. 61/935,634, filed on Feb. 4, 2014.

(51) Int. Cl. C12N 5/0775; A61K 35/28

(52) U.S. Cl. CPC C12N 5/0668; C12N 5/0667; C12N 5/0663; A61K 35/28; C12N 2501/24

(57) ABSTRACT

The invention disclosed provides populations and compositions of mesenchymal stem and progenitor cells derived from adult tissues that are subjected to a process or processes designed to enhance a biological mechanism prior to implantation of the cells for therapeutic use. The processes involve preconditioning of said mesenchymal stem cells with a physiological stressor in vitro, so as to enhance therapeutically significant biological properties. Specific embodiments of the invention include stimulation of angiogenic and immune modulatory properties by preconditioning with interferon-gamma, as well as other physiologically-relevant stressors such as activation of toll like receptors. Immunologically-relevant properties of preconditioned cells including the treatment of cytokine storm associated with sepsis, or viral infection. In the angiogenic embodiment of the invention, cells are used for the treatment of peripheral artery disease, its advanced form, critical limb ischemia, or ischemic heart failure.

### FIELD OF THE INVENTION

The invention pertains to the field of immune modulation, particularly, to the field of cell therapy applied to immune modulation. Furthermore, the field relates to stimulation of angiogenesis utilizing cell therapy.

### BACKGROUND

Immunological tolerance is a cardinal feature of the immune system, allowing for recognition and elimination of pathological threats, while selectively ignoring antigens that belong to the body. Understanding mechanisms of immunological tolerance, and having the ability to induce this process would make a major impact in autoimmune conditions, which affect approximately 8% of the US population. Major autoimmune diseases include rheumatoid arthritis, multiple sclerosis, type 1 diabetes, systemic lupus erythromatosis, and inflammatory bowel disease. Traditionally, autoimmune conditions are treated with non-specific inhibitors of inflammation such as steroids, as well as immune suppressive agents such as cyclosporine, 5-azathrioprine, and methotrexate. These approaches globally suppress immune functions and have numerous undesirable side effects. Unfortunately, given the substantial decrease in quality of life observed in patients with autoimmunity, the potential of alleviation of autoimmune symptoms outweighs the side effects such as opportunistic infections and increased predisposition to neoplasia. The introduction of “biological therapies” such as anti-TNF-alpha antibodies has led to some improvements in prognosis, although side effects are still present due to the non-specific nature of the intervention. Regardless, sales of TNF-alpha inhibitors have been quite successful: Humira ($9.2B; 2012), Enbrel ($7.8B; 2011), Remicade ($6.7B; 2011). These approaches do not “cure” autoimmunity, but merely alleviate symptomology.

To “cure” autoimmunity, it is essential to delete/inactivate the T cell clone that is recognizing the autoantigen in a selective manner. This would be akin to recapitulating the natural process of tolerance induction. While thymic deletion was the original process identified as being responsible for selectively deleting autoreactive T cells, it became clear that numerous redundant mechanisms exist that are not limited to the neonatal period. Specifically, a “minor image” immune system was demonstrated to co-exist with the conventional immune system. Conventional T cells are activated by self-antigens to die in the thymus and conventional T cells that are not activated receive a survival signal [1]; the “mirror image”, T regulatory (Treg) cells are actually selected to live by encounter with self-antigens, and Treg cells that do not bind self antigens are deleted [2, 3]. Thus the self-nonself discrimination by the immune system occurs in part based on self antigens depleting autoreactive T cells, while promoting the generation of Treg cells. An important point for development of an antigen-specific tolerogenic vaccine is that in adult life, and in the periphery, autoreactive T cells are “anergized” by presentation of self-antigens in absence of danger signals, and autoreactive Treg are generated in response to self antigens. Although the process of T cell deletion in the thymus is different than induction of T cell anergy, and Treg generation in the thymus, results in a different type of Treg as compared to peripheral induced Treg, in many aspects, the end result of adult tolerogenesis is similar to that which occurs in the neonatal period.

Specific examples of tolerogenesis that occurs in adults includes settings such as pregnancy, cancer, and oral tolerance. In the situation of pregnancy, studies have demonstrated selective inactivation of maternal T cell clones that recognize fetal antigens occurs through a variety of mechanisms, including FasL expression on fetal and placental cells [4], antigen presentation in the context of PD1-L [5], and HLA-G interacting with immune inhibitory receptors such as ILT4 [6]. In pregnancy, “tolerogenic antigen presentation” occurs only through the indirect pathway of antigen presentation [7]. Other pathways of selective tolerogenesis in pregnancy include the stimulation of Treg cells, which have been demonstrated essential for successful pregnancy [8]. In the context of cancer, depletion of tumor specific T cells, while sparing of T cells with specificities to other antigens has been demonstrated by the tumor itself or tumor associated cells [9-12]. Additionally, Treg cells have been demonstrated to actively suppress anti-tumor T cells, perhaps as a “back up” mechanism of tumor immune evasion [13-15]. At a clinical level the ability of tumors to inhibit peripheral T cell activity has been associated in numerous studies with poor prognosis [16-18]. Oral tolerance is the process by which ingested antigens induce generation of antigen-specific TGF-beta producing cells (called “Th3” by some) [19-21], as well as Treg cells [22, 23]. Ingestion of antigen, including the autoantigen collagen II [24], has been shown to induce inhibition of both T and B cell responses in a specific manner [25, 26]. It appears that induction of regulatory cells, as well as deletion/anergy of effector cells is associated with antigen presentation in a tolerogenic manner [27]. Remission of disease in animal models of RA [28], multiple sclerosis [29], and type I diabetes [30], has been reported by oral administration of autoantigens. Furthermore, clinical trials have shown signals of efficacy of oral tolerance in autoimmune diseases such as rheumatoid arthritis [31], autoimmune uveitis [32], and multiple sclerosis [33]. In all of these natural conditions of tolerance, common molecules and mechanisms seem to be operating. Accordingly, a natural means of inducing tolerance would be the administration of a “universal donor” cell with tolerogenic potential that generate molecules similar to those found in physiological conditions of tolerance induction.

While use of MSC have been applied therapeutically in a variety of autoimmune conditions, responses have been variable. The current invention provides means to condition MSC before administration, in order to endow enhanced immune modulatory or angiogenic activities.

### DESCRIPTION OF THE INVENTION

The invention provides means of augmenting immune modulatory and angiogenic properties of MSC through preconditioning with agents that induce a stress response in MSC. Previous clinical utilization of MSC involves administration of cultured cells. While culture under defined conditions allows for expansion of cells, which in many times are administered without differentiation. While artificially generated MSC are usually administered after in vitro culture, their migratory route from administration site to target site for therapeutic activity involves various biological stimuli, as well as locations of anatomical sequestration such as the spleen, liver, or lung. Accordingly, the invention provides means to “prime” MSC in vitro before administration, for augmentation of homing, immune modulatory, and therapeutic activity.

“Treat” or “treatment” means improving the symptoms and ameliorating autoimmune, septic, or pulmonary disease. Additionally, “treat” means improving ischemic conditions. Methods for measuring the rate of “treatment” efficacy are known in the art and include, for example, assessment of inflammatory cytokines.

“Angiogenesis” means any alteration of an existing vascular bed or the formation of new vasculature which benefits tissue perfusion. This includes the formation of new vessels by sprouting of endothelial cells from existing blood vessels or the remodeling of existing vessels to alter size, maturity, direction or flow properties to improve blood perfusion of tissues. As used herein the terms, “angiogenesis,” “revascularization,” “increased collateral circulation,” and “regeneration of blood vessels” are considered as synonymous.

“Chronic wound” means a wound that has not completely closed in twelve weeks since the occurrence of the wound in a patient having a condition, disease or therapy associated with defective healing. Conditions, diseases or therapies associated with defective healing include, for example, diabetes, arterial insufficiency, venous insufficiency, chronic steroid use, cancer chemotherapy, radiotherapy, radiation exposure, and malnutrition. A chronic wound includes defects resulting in inflammatory excess (e.g., excessive production of Interleukin-6 (IL-6), tumor necrosis factor-alpha (TNF-.alpha.), and MMPs), a deficiency of important growth factors needed for proper healing, bacterial overgrowth and senescence of fibroblasts. A chronic wound has an epithelial layer that fails to cover the entire surface of the wound and is subject to bacterial colonization.

“Therapeutically effective amount” means the amount of cells, conditioned media or exosomes that, when administered to a mammal for treating a chronic wound, or angiogenic insufficiency is sufficient to effect such treatment. The “therapeutically effective amount” may vary depending on the size of the wound, and the age, weight, physical condition and responsiveness of the mammal to be treated.

“Therapeutic agent” means to have “therapeutic efficacy” in modulating angiogenesis and/or wound healing and an amount of the therapeutic is said to be a “angiogenic modulatory amount”, if administration of that amount of the therapeutic is sufficient to cause a significant modulation (i.e., increase or decrease) in angiogenic activity when administered to a subject (e.g., an animal model or human patient) needing modulation of angiogenesis.

“Growth factor” can be a naturally occurring, endogenous or exogenous protein, or recombinant protein, capable of stimulating cellular proliferation and/or cellular differentiation and cellular migration.

“About” or “approximately” means within an acceptable range for the particular value as determined by one of ordinary skill in the art, which will depend in part on how the value is measured or determined, e.g., the limitations of the measurement system. For example, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Unless otherwise stated, the term ‘about’ means within an acceptable error range for the particular value.

“Pharmaceutically acceptable” refers to a natural or synthetic substance means that the substance has an acceptable toxic effect in view of its much greater beneficial effect, while the related the term, “physiologically acceptable,” means the substance has relatively low toxicity.

“Endothelial cell mitogen” means any protein, polypeptide, variant or portion thereof that is capable of, directly or indirectly, inducing endothelial cell growth. Such proteins include, for example, acidic and basic fibroblast growth factors (aFGF) (GenBank Accession No. NP.sub.-149127) and bFGF (GenBank Accession No. AAA52448), vascular endothelial growth factor (VEGF) (GenBank Accession No. AAA35789 or NP.sub.-001020539), epidermal growth factor (EGF) (GenBank Accession No. NP.sub.-001954), transforming growth factor .alpha. (TGF-.alpha.) (GenBank Accession No. NP.sub.-003227) and transforming growth factor.beta. (TFG-.beta.) (GenBank Accession No. 1109243A), platelet-derived endothelial cell growth factor (PD-ECGF) (GenBank Accession No. NP.sub.-001944), platelet-derived growth factor (PDGF) (GenBank Accession No. 1109245A), tumor necrosis factor.alpha. (TNF-.alpha.) (GenBank Accession No. CAA26669), hepatocyte growth factor (HGF) (GenBank Accession No. BAA14348), insulin like growth factor (IGF) (GenBank Accession No. P08833), erythropoietin (GenBank Accession No. P01588), colony stimulating factor (CSF), macrophage-CSF (M-CSF) (GenBank Accession No. AAB59527), granulocyte/macrophage CSF (GM-CSF) (GenBank Accession No. NP.sub.-000749), monocyte chemotactic protein-1 (GenBank Accession No. P13500) and nitric oxide synthase (NOS) (GenBank Accession No. AAA36365). See, Klagsbrun, et al., Annu. Rev. Physiol., 53:217-239 (1991); Folkman, et al., J. Biol. Chem., 267:10931-10934 (1992) and Symes, et al., Current Opinion in Lipidology, 5:305-312 (1994). Variants or fragments of a mitogen may be used as long as they induce or promote endothelial cell or endothelial progenitor cell growth. Preferably, the endothelial cell mitogen contains a secretory signal sequence that facilitates secretion of the protein. Proteins having native signal sequences, e.g., VEGF, are preferred. Proteins that do not have native signal sequences, e.g., bFGF, can be modified to contain such sequences using routine genetic manipulation techniques. See, Nabel et al., Nature, 362:844 (1993).

“Mesenchymal stem cell” or “MSC” refers to cells that are (1) adherent to plastic, (2) express CD73, CD90, and CD105 antigens, while being CD14, CD34, CD45, and HLA-DR negative, and (3) possess ability to differentiate to osteogenic, chondrogenic and adipogenic lineage. As used herein, “mesenchymal stromal cell” or “MSC” can be derived from any tissue including, but not limited to, bone marrow, adipose tissue, amniotic fluid, endometrium, trophoblast-derived tissues, cord blood, Wharton jelly, placenta, amniotic tissue, derived from pluripotent stem cells, and tooth. As used herein, “mesenchymal stromal cell” or “MSC” includes cells that are CD34 positive upon initial isolation from tissue but are similar to cells described about phenotypically and functionally. As used herein, “MSC” includes cells that are isolated from tissues using cell surface markers selected from the list comprised of NGF-R, PDGF-R, EGF-R, IGF-R, CD29, CD49a, CD56, CD63, CD73, CD105, CD106, CD140b, CD146, CD271, MSCA-1, SSEA4, STRO-1 and STRO-3 or any combination thereof, and satisfy the ISCT criteria either before or after expansion. As used herein, “mesenchymal stromal cell” or “MSC” includes cells described in the literature as bone marrow stromal stem cells (BMSSC), marrow-isolated adult multipotent inducible cells (MIAMI) cells, multipotent adult progenitor cells (MAPC), mesenchymal adult stem cells (MASCS), MultiStem®, Prochymal®, remestemcel-L, Mesenchymal Precursor Cells (MPCs), Dental Pulp Stem Cells (DPSCs), PLX cells, PLX-PAD, AlloStem®, Astrostem®, Ixmyelocel-T, MSC-NTF, NurOwn™, Stemedyne™-MSC, Stempeucel®, StempeucelCLI, StempeucelOA, HiQCell, Hearticellgram-AMI, Revascor®, Cardiorel®, Cartistem®, Pneumostem®, Promostem®, Homeo-GH, AC607, PDA001, SB623, CX601, AC607, Endometrial Regenerative Cells (ERC), adipose-derived stem and regenerative cells (ADRCs).

The preferred animal subject of the present invention is a mammal. By the term “mammal” is meant an individual belonging to the class Mammalia. The invention is particularly useful in the treatment of human subjects.

The present invention provides for methods of treatment of acute respiratory distress syndrome and cytokine storm, which methods comprise administering to a subject in need of such treatment a therapeutically effective amount of MSC that have been subjected to therapeutic preconditioning, combinations of MSC with monocytes, and conditioned media from MSC or MSC combined with monocytes. In one particular embodiment exosomes are purified from MSC or MSC combined with monocytes.

While it is possible to use a composition provided by the present invention for therapy as is, it may be preferable to administer it in a pharmaceutical formulation, e.g., in admixture with a suitable pharmaceutical excipient, diluent, or carrier selected with regard to the intended route of administration and standard pharmaceutical practice. Accordingly, in one aspect, the present invention provides a pharmaceutical composition or formulation comprising at least one active composition, or a pharmaceutically acceptable derivative thereof, in association with a pharmaceutically acceptable excipient, diluent, and/or carrier. The excipient, diluent and/or carrier must be “acceptable” in the sense of being compatible with the other ingredients of the formulation and not deleterious to the recipient thereof.

In one embodiment, MSC are generated from bone marrow mononuclear cells (BM MNC). Alternatively, other tissue sources can be used including adipose tissue, peripheral blood, amniotic membrane, umbilical cord blood or Whartons Jelly. The culture method for generating begins with the enrichment of BM MNC from the starting material (e.g., tissue) by removing red blood cells and some of the polynucleated cells using a conventional cell fractionation method. For example, cells are fractionated by using a FICOLL.®. density gradient separation. The volume of starting material needed for culture is typically small, for example, 40 to 50 mL, to provide a sufficient quantity of cells to initiate culture. However, any volume of starting material may be used. Nucleated cell concentration is then assessed using an automated cell counter, and the enriched fraction of the starting material is inoculated into a biochamber (cell culture container). The number of cells inoculated into the biochamber depends on its volume. Prior to inoculation, a biochamber is primed with culture medium. Illustratively, the medium used in accordance with the invention comprises three basic components. The first component is a media component comprised of IMDM, MEM, DMEM, RPMI 1640, Alpha Medium or McCoy's Medium, or an equivalent known culture medium component. The second is a serum component which comprises at least horse serum or human serum and may optionally further comprise fetal calf serum, newborn calf serum, and/or calf serum. Optionally, serum free culture mediums known in the art may be used. The third component is a corticosteroid, such as hydrocortisone, cortisone, dexamethasone, solumedrol, or a combination of these, preferably hydrocortisone. The cells and media are then passed through the biochamber at a controlled ramped perfusion schedule during culture process. The cells are cultures for 2, 4, 6, 8, 10, 12, 14, 16 or more days. Preferably, the cells are cultured for 12 days. These cultures are typically carried out at a pH which is roughly physiologic, i.e. 6.9 to 7.6. The medium is kept at an oxygen concentration that corresponds to an oxygen-containing atmosphere which contains from 1 to 20 vol. percent oxygen, preferably 3 to 12 vol. percent oxygen. The preferred range of 0.sub.2 concentration refers to the concentration of 0.sub.2 near the cells, not necessarily at the point of 0.sub.2 introduction which may be at the medium surface or through a membrane.

Standard culture schedules call for medium and serum to be exchanged weekly, either as a single exchange performed weekly or a one-half medium and serum exchange performed twice weekly. Preferably, the nutrient medium of the culture is replaced, preferably perfused, either continuously or periodically, at a rate of about 1 ml per ml of culture per about 24 to about 48 hour period, for cells cultured at a density of from 2.times.10.sup.6 to 1.times.10.sup.7 cells per ml. For cell densities of from 1.times.10.sup.4 to 2.times.10.sup.6 cells per ml the same medium exchange rate may be used. Thus, for cell densities of about 10.sup.7 cells per ml, the present medium replacement rate may be expressed as 1 ml of medium per 10.sup.7 cells per about 24 to about 48 hour period. For cell densities higher than 10.sup.7 cells per ml, the medium exchange rate may be increased proportionality to achieve a constant medium and serum flux per cell per unit time.

A method for culturing bone marrow cells is described in Lundell, et al., “Clinical Scale Expansion of Cryopreserved Small Volume Whole Bone Marrow Aspirates Produces Sufficient Cells for Clinical Use,” J. Hematotherapy (1999) 8:115-127 (which is incorporated herein by reference). Bone marrow (BM) aspirates are diluted in isotonic buffered saline (Diluent 2, Stephens Scientific, Riverdale, N.J.), and nucleated cells are counted using a Coulter ZM cell counter (Coulter Electronics, Hialeah, Fla.). Erythrocytes (non-nucleated) are lysed using a Manual Lyse (Stephens Scientific), and mononuclear cells (MNC) are separated by density gradient centrifugation (Ficoll-Paque.®. Plus, Pharmacia Biotech, Uppsala, Sweden) (specific gravity 1.077) at 300 g for 20 min at 25.degree. C. BM MNC are washed twice with long-term BM culture medium (LTBMC) which is Iscove's modified Dulbecco's medium (IMDM) supplemented with 4 mM L-glutamine 9GIBCO BRL, Grand Island, N.Y.), 10% fetal bovine serum (FBS), (Bio-Whittaker, Walkersville, Md.), 10% horse serum (GIBCO BRL), 20 .mu.g/ml vancomycin (Vancocin.®. HCl, Lilly, Indianapolis, Ind.), 5 gentamicin (Fujisawa USA, Inc., Deerfield, Ill.), and 5 .mu.M hydrocortisone (Solu-Cortef.®, Upjohn, Kalamazoo, Mich.) before culture.

### EXAMPLES

### Example 1

### Reduction in Endotoxin Induced Mortality by Administration of IFN-gamma Treated Wharton's Jelly MSC

Human umbilical cords were obtained from full term women immediately after delivery. After thorough washing with phosphate buffered saline containing penicillin and streptomycin, the cord was cut open and the arteries and veins were removed. The tissue was treated with collagenease blend at a net concentration of 500 μg/ml for 20 hr. Post-digestion with collagenease the cells were filtered through a 100μ cell strainer to remove tissue debris. Cells were then washed with PBS and cultured in KO-DMEM containing 10% FBS, 2 mM L-Glutamine and antibiotics. WJSCs were grown till confluence and cells were used at Passage 8 for experimentation. To stimulate a stress response, MSC were cultured for 48 hours in 150 IU/ml of interferon gamma (IFN-MSC) or in control DMEM media (Control-MSC).

Female BALB/c mice were treated with 20 mg/kg of LPS diluted in 100 μL 0.9% saline solution, administered by intraperitoneal (IP) injection in a single dose. All groups consisted of 10 mice per group. Group 1 was control, which received LPS alone. Group 2 received MSC, 500,000 cells per mouse, administered intravenously. Group 3 received IFN-MSC, 500,000 per mouse, administered intravenously. Mice were followed for 5 days. Administration of cells was performed 6 hours after endotoxin challenge. As seen in the Table below, control treated animals receiving LPS alone had a mortality of 9/10, MSC treated animals had a mortality of 8/10, and IFN-MSC had a mortality of 2/10, when assessed at day 5 post endotoxin challenge.

| | | | | | | |
|---|---|---|---|---|---|---|
| Group and | | | | | | |
| Mouse | Day 1 | Day 1 | | | | |
| Number | (12 hours) | (24 hours) | Day 2 | Day 3 | Day 4 | Day 5 |

Control (LPS Alone)

| | | | | | | |
|---|---|---|---|---|---|---|
| 1 | Alive | Alive | Dead | Dead | Dead | Dead |
| 2 | Alive | Dead | Dead | Dead | Dead | Dead |
| 3 | Dead | Dead | Dead | Dead | Dead | Dead |
| 4 | Alive | Dead | Dead | Dead | Dead | Dead |
| 5 | Alive | Dead | Dead | Dead | Dead | Dead |
| 6 | Alive | Dead | Dead | Dead | Dead | Dead |
| 7 | Dead | Dead | Dead | Dead | Dead | Dead |
| 8 | Alive | Dead | Dead | Dead | Dead | Dead |
| 9 | Alive | Dead | Dead | Dead | Dead | Dead |
| 10 | Alive | Alive | Alive | Alive | Alive | Alive |

MSC (LPS + MSC)

| | | | | | | |
|---|---|---|---|---|---|---|
| 11 | Alive | Dead | Dead | Dead | Dead | Dead |
| 12 | Alive | Dead | Dead | Dead | Dead | Dead |
| 13 | Alive | Dead | Dead | Dead | Dead | Dead |
| 14 | Dead | Dead | Dead | Dead | Dead | Dead |
| 15 | Alive | Alive | Alive | Alive | Alive | Alive |
| 16 | Alive | Alive | Alive | Alive | Alive | Alive |
| 17 | Dead | Dead | Dead | Dead | Dead | Dead |
| 18 | Dead | Dead | Dead | Dead | Dead | Dead |
| 19 | Alive | Alive | Alive | Alive | Alive | Alive |
| 20 | Dead | Dead | Dead | Dead | Dead | Dead |

IFN-MSC

| | | | | | | |
|---|---|---|---|---|---|---|
| 21 | Alive | Alive | Alive | Alive | Alive | Alive |
| 22 | Alive | Alive | Alive | Alive | Alive | Alive |
| 23 | Alive | Alive | Alive | Alive | Alive | Alive |
| 24 | Alive | Alive | Alive | Alive | Alive | Alive |
| 25 | Alive | Alive | Alive | Alive | Alive | Alive |
| 26 | Alive | Alive | Alive | Alive | Alive | Alive |
| 27 | Alive | Alive | Dead | Dead | Dead | Dead |
| 28 | Alive | Alive | Dead | Dead | Dead | Dead |
| 29 | Alive | Alive | Alive | Alive | Alive | Alive |
| 30 | Alive | Alive | Alive | Alive | Alive | Alive |

### REFERENCES

1. Ramsdell, F. and B. J. Fowlkes, Clonal deletion versus clonal anergy: the role of the thymus in inducing self tolerance. Science, 1990. 248(4961): p. 1342-8.

2. Apostolou, I., et al., Origin of regulatory T cells with known specificity for antigen. Nat Immunol, 2002. 3(8): p. 756-63.

3. Aschenbrenner, K., et al., Selection of Foxp3+ regulatory T cells specific for self antigen expressed and presented by Aire+ medullary thymic epithelial cells. Nat Immunol, 2007. 8(4): p. 351-8.

4. Vacchio, M. S. and R. J. Hodes, Fetal expression of Fas ligand is necessary and sufficient for induction of CD8 T cell tolerance to the fetal antigen H-Y during pregnancy. J Immunol, 2005. 174(8): p. 4657-61.

5. D'Addio, F., et al., The link between the PDL1 costimulatory pathway and Th17 in fetomaternal tolerance. J Immunol, 2011. 187(9): p. 4530-41.

6. Kuroki, K. and K. Maenaka, Immune modulation of HLA-G dimer in maternal-fetal interface. Eur J Immunol, 2007. 37(7): p. 1727-9.

7. Erlebacher, A., et al., Constraints in antigen presentation severely restrict T cell recognition of the allogeneic fetus. J Clin Invest, 2007. 117(5): p. 1399-411.

8. Chen, T., et al., Self-specific memory regulatory T cells protect embryos at implantation in mice. J Immunol, 2013. 191(5): p. 2273-81.

9. Harimoto, H., et al., Inactivation of tumor-specific CD8(+) CTLs by tumor-infiltrating tolerogenic dendritic cells. Immunol Cell Biol, 2013. 91(9): p. 545-55.

10. Ney, J. T., et al., Autochthonous liver tumors induce systemic T cell tolerance associated with T cell receptor down-modulation. Hepatology, 2009. 49(2): p. 471-81.

11. Cheung, A. F., et al., Regulated expression of a tumor-associated antigen reveals multiple levels of T-cell tolerance in a mouse model of lung cancer. Cancer Res, 2008. 68(22): p. 9459-68.

12. Bai, A., et al., Rapid tolerization of virus-activated tumor-specific CD8+ T cells in prostate tumors of TRAMP mice. Proc Natl Acad Sci USA, 2008. 105(35): p. 13003-8.

13. Jacobs, J. F., et al., Regulatory T cells in melanoma: the final hurdle towards effective immunotherapy? Lancet Oncol, 2012. 13(1): p. e32-42.

14. Pedroza-Gonzalez, A., et al., Activated tumor-infiltrating CD4+ regulatory T cells restrain antitumor immunity in patients with primary or metastatic liver cancer. Hepatology, 2013. 57(1): p. 183-94.

15. Donkor, M. K., et al., T cell surveillance of oncogene-induced prostate cancer is impeded by T cell-derived TGF-betal cytokine. Immunity, 2011. 35(1): p. 123-34.

16. Whiteside, T. L., Down-regulation of zeta-chain expression in T cells: a biomarker of prognosis in cancer? Cancer Immunol Immunother, 2004. 53(10): p. 865-78.

17. Whiteside, T. L., Signaling defects in T lymphocytes of patients with malignancy. Cancer Immunol Immunother, 1999. 48(7): p. 346-52.

18. Reichert, T. E., et al., Signaling abnormalities, apoptosis, and reduced proliferation of circulating and tumor-infiltrating lymphocytes in patients with oral carcinoma. Clin Cancer Res, 2002. 8(10): p. 3137-45.

19. Faria, A. M. and H. L. Weiner, Oral tolerance. Immunol Rev, 2005. 206: p. 232-59.

20. Weiner, H. L., Induction and mechanism of action of transforming growth factor-beta-secreting Th3 regulatory cells. Immunol Rev, 2001. 182: p. 207-14.

21. Fukaura, H., et al., Induction of circulating myelin basic protein and proteolipid protein-specific transforming growth factor-beta1-secreting Th3 T cells by oral administration of myelin in multiple sclerosis patients. J Clin Invest, 1996. 98(1): p. 70-7.

22. Palomares, O., et al., Induction and maintenance of allergen-specific FOXP3+ Treg cells in human tonsils as potential first-line organs of oral tolerance. J Allergy Clin Immunol, 2012. 129(2): p. 510-20, 520 el-9.

23. Yamashita, H., et al., Overcoming food allergy through acquired tolerance conferred by transfer of Tregs in a murine model. Allergy, 2012. 67(2): p. 201-9.

24. Park, K. S., et al., Type II collagen oral tolerance; mechanism and role in collagen-induced arthritis and rheumatoid arthritis. Mod Rheumatol, 2009.

25. Womer, K. L., et al., A pilot study on the immunological effects of oral administration of donor major histocompatibility complex class II peptides in renal transplant recipients. Clin Transplant, 2008. 22(6): p. 754-9.

26. Faria, A. M. and H. L. Weiner, Oral tolerance: therapeutic implications for autoimmune diseases. Clin Dev Immunol, 2006. 13(2-4): p. 143-57.

27. Park, M. J., et al., A distinct tolerogenic subset of splenic IDO(+)CD11b(+) dendritic cells from orally tolerized mice is responsible for induction of systemic immune tolerance and suppression of collagen-induced arthritis. Cell Immunol, 2012. 278(1-2): p. 45-54.

28. Thompson, H. S., et al., Suppression of collagen induced arthritis by oral administration of type II collagen: changes in immune and arthritic responses mediated by active peripheral suppression. Autoimmunity, 1993. 16(3): p. 189-99.

29. Song, F., et al., The thymus plays a role in oral tolerance induction in experimental autoimmune encephalomyelitis. Ann NY Acad Sci, 2004. 1029: p. 402-4.

30. Hanninen, A. and L. C. Harrison, Mucosal tolerance to prevent type 1 diabetes: can the outcome be improved in humans? Rev Diabet Stud, 2004. 1(3): p. 113-21.

31. Wei, W., et al., A multicenter, double-blind, randomized, controlled phase III clinical trial of chicken type II collagen in rheumatoid arthritis. Arthritis Res Ther, 2009. 11(6): p. R180.

32. Thurau, S. R., et al., Molecular mimicry as a therapeutic approach for an autoimmune disease: oral treatment of uveitis patients with an MHC-peptide crossreactive with autoantigen-first results. Immunol Lett, 1997. 57(1-3): p. 193-201.

33. Weiner, H. L., et al., Double-blind pilot trial of oral tolerization with myelin antigens in multiple sclerosis. Science, 1993. 259(5099): p. 1321-4.

1. A mesenchymal stem cell with augmented immune modulatory activity, said augmentation induced by exposure to an agent or plurality of agents inducing a stress response in said mesenchymal stem cell.

2. The mesenchymal stem cell of claim 1, wherein said immune modulatory activity is ability to inhibit cytokine storm in a patient.

3. The mesenchymal stem cell of claim 2, wherein said immune modulatory activity is ability to inhibit cytokine storm in a mammal.

4. The mesenchymal stem cell of claim 2, wherein said cytokine storm is an elevation of at least 50% compared to basal state of one or more cytokines, selected from a group of cytokines comprising of: a) interferon alpha; b) interferon gamma; c) TNF-alpha; d) IL-1; e) IL-6; f) IL-12; g) IL-18; h) IL-21; i) IL-17; k) IL-33; and 1) HMGB-1.

5. The mesenchymal stem cell of claim 2, wherein said immune modulatory activity comprises ability of said mesenchymal stem cell to inhibit pathological neutrophil accumulation in the lung.

6. The mesenchymal stem cell of claim 2, wherein said immune modulatory activity comprises ability of said mesenchymal stem cell to inhibit vascular leak syndrome in response to inflammatory stimuli.

7. The mesenchymal stem cell of claim 2, wherein said immune modulatory activity comprises ability of said mesenchymal stem cell to inhibit multiorgan failure.

8. The mesenchymal stem cell of claim 2, wherein said immune modulatory activity comprises ability of said mesenchymal stem cell to inhibit disseminated intravascular coagulation.

9. The mesenchymal stem cell of claim 1, wherein said agent capable of eliciting a stress response is characterized by ability to induce an upregulation of transcription of more than 15 percent as compared to baseline, in said mesenchymal stem cell, of factors selected from a group comprising of: a) nuclear factor kappa B (NF-kB); b) hypoxia inducible factor alpha (HIF-1alpha), c) hemoxygenase-1 (HO-1); d) indolamine 2,3 deoxygenase; and e) heat shock protein 65 (hsp-65).

10. The mesenchymal stem cell of claim 1, wherein said agent capable of stimulating a stress response is selected from a group comprising of: a) interferon gamma; b) IVIG; c) monocyte conditioned media; d) supernatant from neutrophil extracellular trap exposed peripheral blood mononuclear cells; e) coculture with monocytes; f) coculture with monocytes that have been pretreated with IVIG; g) coculture with T cells; h) coculture with T cells that have been exposed to a T cell stimulus; i) coculture with NK cells; j) peptidoglycan isolated from gram positive bacteria; k) lipoteichoic acid isolated from gram positive bacteria; l) lipoprotein isolated from gram positive bacteria; m) lipoarabinomannan isolated from mycobacteria, n) zymosan isolated from yeast cell well; o) Polyadenylic-polyuridylic acid; p) poly (IC); q) lipopolysaccharide; r) monophosphoryl lipid A; s)flagellin; t) Gardiquimod; u) Imiquimod; v) R848; w) oligonucleosides containing CpG motifs; and x) 23S ribosomal RNA.

11. The mesenchymal stem cell of claim 1, wherein said mesenchymal stem cell is selected from a group of mesenchymal stem cells comprising of: a) adipose mesenchymal stem cells; b) bone marrow mesenchymal stem cells; c) umbilical cord mesenchymal stem cells; d) Wharton's jelly mesenchymal stem cells; e) tooth mesenchymal stem cells; f) amnionic mesenchymal stem cells; g) placental mesenchymal stem cells; h) circulating blood mesenchymal stem cells and i) hair follicle mesenchymal stem cells.

12. The mesenchymal stem cell of claim 11, wherein said adipose tissue mesenchymal stem cells express one or more markers selected from a group comprising of: CD13, CD29, CD44, CD63, CD73, CD90, CD166, Aldehyde dehydrogenase (ALDH), and ABCG2.

13. The mesenchymal stem cell of claim 11, wherein said adipose tissue derived mesenchymal stem cells are a population of purified mononuclear cells extracted from adipose tissue capable of proliferating in culture for more than 1 month.

14. The mesenchymal stem cell of claim 11, wherein said bone marrow mesenchymal stem cells are derived from bone marrow mononuclear cells.

15. The mesenchymal stem cell of claim 11, wherein said bone marrow mesenchymal stem cells are selected based on the ability to differentiate into one or more of the following cell types: bone; cartilage; or adipose tissue, subsequent to exposure to differentiation stimuli.

16. The mesenchymal stem cell of claim 11, wherein said bone marrow mesenchymal stem cells are selected based on expression of one or more of the following antigens: CD90, c-kit, flk-1, Stro-1, CD105, CD73, CD31, CD146, vascular endothelial-cadherin, CD133 and CXCR-4.

17. The mesenchymal stem cell of claim 11, wherein said bone marrow mesenchymal stem cells are enriched for expression of CD90 and CD73.

18. The mesenchymal stem cell of claim 11, wherein said bone marrow mesenchymal stem cells lack significant expression of CD34.

19. The mesenchymal stem cell of claim 11, wherein said placental mesenchymal stem cells are identified based on expression of one or more antigens selected from a group comprising: Oct-4, Rex-1, CD9, CD13, CD29, CD44, CD166, CD90, CD105, SH-3, SH-4, TRA-1-60, TRA-1-81, SSEA-4 and Sox-2.

20. The mesenchymal stem cell of claim 11, wherein said amniotic fluid mesenchymal stem cells are isolated by introduction of a fluid extraction means into the amniotic cavity under ultrasound guidance.

21. The mesenchymal stem cell of claim 11, wherein said amniotic fluid mesenchymal stem cells are selected based on expression of one or more of the following antigens: SSEA3, SSEA4, Tra-1-60, Tra-1-81, Tra-2-54, HLA class I, CD13, CD44, CD49b, CD105, Oct-4, Rex-1, DAZL and Runx-1.

22. The mesenchymal stem cell of claim 11, wherein said amniotic fluid mesenchymal stem cells are selected based on lack of expression of one or more of the following antigens: CD34, CD45, and HLA Class II.

23. The mesenchymal stem cell of claim 11, wherein said circulating peripheral blood mesenchymal stem cells are characterized by ability to proliferate in vitro for a period of over 1 months.

24. The mesenchymal stem cell of claim 11, wherein said circulating peripheral blood mesenchymal stem cells are characterized by expression of CD34, CXCR4, CD117, CD113, and c-met.

25. The mesenchymal stem cell of claim 11, wherein said circulating peripheral blood mesenchymal stem cells lack substantial expression of differentiation associated markers.

26. The mesenchymal stem cell of claim 25, wherein said differentiation associated markers are selected from a group comprising of CD2, CD3, CD4, CD11, CD11a, Mac-1, CD14, CD16, CD19, CD24, CD33, CD36, CD38, CD45, CD56, CD64, CD68, CD86, CD66b, and HLA-DR.

27. The mesenchymal stem cell of claim 11, wherein said mesenchymal stem cells express one or more of the following markers: STRO-1, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1.

28. The mesenchymal stem cell of claim 11, wherein said mesenchymal stem cells do not express substantial levels of HLA-DR, CD117, and CD45.

29. The mesenchymal stem cell of claim 1, wherein said cells are Wharton's Jelly derived mesenchymal stem cells, said cells treated with interferon gamma for a period of time from 1 hour to 160 hours.

30. The mesenchymal stem cell of claim 1, wherein said cells are Wharton's Jelly derived mesenchymal stem cells, said cells treated with interferon gamma for a period of time from 3 hours to 72 hours.

31. The mesenchymal stem cell of claim 1, wherein said cells are Wharton's Jelly derived mesenchymal stem cells, said cells treated with interferon gamma for a period of time for approximately 48 hours.

32. The mesenchymal stem cell of claim 1, wherein said cells are Wharton's Jelly derived mesenchymal stem cells, said cells treated with interferon gamma at a total concentration ranging from 20-1000 IU per ml.

33. The mesenchymal stem cell of claim 1, wherein said cells are Wharton's Jelly derived mesenchymal stem cells, said cells treated with interferon gamma at a total concentration ranging from 50-500 IU per ml.

34. The mesenchymal stem cell of claim 1, wherein said cells are Wharton's Jelly derived mesenchymal stem cells, said cells treated with interferon gamma at a total concentration of approximately 150 IU per ml.

35. A method of treating cytokine storm associated with a viral infection, comprising administration of population of mesenchymal stem cells that have been preconditioned with a stress inducing stimuli.

36. The method of claim 35, wherein said stress inducing stimuli is interferon gamma.

37. The method of claim 35, wherein said stress inducing stimuli comprises exposure to a culture condition selected from a group consisting of: a) ozone or ozonized media; b) hydrogen peroxide; and c) a low pH.
